(19) United States
(12) Patent Application Publication
(10) Pub. No.: US 2024/0191060 A1
(43) Pub. Date: Jun. 13, 2024

(54) RUBBER MIXTURES CONTAINING N,N'-DIALKYL-P-PHENYLENEDIAMINES

(71) Applicant: LANXESS Deutschland GmbH, Cologne (DE)

(72) Inventors: Olaf Halle, Cologne (DE); Hermann-Josef Weidenhaupt, Pulheim (DE); Melanie Wiedemeier-Jarad, Dormagen (DE); Achim Schneider, Wuerzburg (DE)

(73) Assignee: LANXESS Deutschland GmbH, Cologne (DE)

(21) Appl. No.: 18/282,834

(22) PCT Filed: Mar. 30, 2022
(86) PCT No.: PCT/EP2022/058458

§ 371 Date: Sep. 19, 2023

(30) Foreign Application Priority Data
Apr. 1, 2021 (EP) 21166705.0
Feb. 7, 2022 (EP) 22155332.4

(51) Int. Cl. C08K 5/18; B60C 1/00; C07C 211/26; C08K 3/36; C08K 5/00; C08K 5/07; C08K 5/13; C08K 5/31; C08K 5/40; C08K 5/5398; C08K 5/548

(52) U.S. Cl. CPC C08K 5/18; C07C 211/26; C08K 3/36; C08K 5/0025; C08K 5/07; C08K 5/13; C08K 5/31; C08K 5/40; C08K 5/5398; C08K 5/548; B60C 1/00; C08K 2201/019

(57) ABSTRACT

New rubber mixtures on the basis of natural rubber and N, N-dicyclohexyl-p-phenylenediamine are extremely well suited for the production of vulcanized rubbers and moulded parts obtainable therefrom, in particular tires, tire parts or technical rubber articles with improved use properties.

The present invention relates to novel rubber mixtures based on natural rubber and N,N′-dicyclohexyl-p-phenylenediamine, to processes for production thereof and to the use thereof for production of rubber vulcanizates by the vulcanization process, and to shaped bodies obtainable therefrom, especially tires, tire parts and industrial rubber articles, and to the use of N,N′-dicyclohexyl-p-phenylenediamine as aging stabilizers for rubber mixtures and rubber vulcanizates based on natural rubber.

The invention of the vulcanization of natural rubber and of synthetic rubber provided a novel material having a unique profile of properties that has contributed substantially to the development of modern technology.

However, natural rubber and synthetic diene rubbers contain double bonds. Specifically the reactive sites thereof in allyl position promote reaction with oxidizing agents such as ozone or oxygen. In the course of what is called aging, for example by contact with oxygen, the whole volume of the rubber article is oxidized, which can be associated with hardening, embrittlement and cracking at the surface of the article, up to and including complete destruction of the article.

It is therefore common practice to protect rubber vulcanizates against destructive environmental influences by means of aging stabilizers. For example, known phenolic, aminic, sulfur-containing or phosphorus-containing aging stabilizers are suitable for improvement of oxygen, ozone, heat and storage stability of rubber vulcanizates.

The best-known aging stabilizers for vulcanizates, made both from natural rubber and from synthetic rubbers, are compounds from the class of the N,N′-dialkyl-p-phenylenediamines and the N-aryl-N′-alkylphenylenediamines. Especially for use in natural rubbers, N-(1,3-dimethylbutyl)-N′-phenyl-p-phenylenediamine (6PPD) and N,N′-bis(1,4-dimethyl)pentyl-p-phenylenediamine (77PD) have become established on the market.

U.S. Pat. No. 3,304,285 discloses that vulcanizates based on SBR rubbers can be protected against the influence of ozone with a synergistic mixture of N,N′-dicyclohexylphenylenediamine and particular N-phenyl-N′-alkylphenylenediamines.

A disadvantage of these known aging stabilizers is the high volatility thereof in the vulcanization of the rubber mixtures.

There was therefore a need for alternative aging stabilizers for rubber vulcanizates based on natural rubber that have a good spectrum of action and simultaneously have reduced volatility in the processing of the natural rubber.

It was an object of the present invention to provide an alternative aging stabilizer for natural rubber that overcomes the disadvantages of the prior art.

It has been found that N,N′-dicyclohexyl-p-phenylenediamine is of excellent suitability for protection of vulcanizates based on natural rubbers against aging processes caused by the action of oxygen and ozone.

Surprisingly, the rubber vulcanizates protected with N,N′-dicyclohexyl-p-phenylenediamine also feature improved performance properties such as tensile strength and elongation at break, and faster full vulcanization. The emission of volatile constituents in the production of the vulcanizates with the aging stabilizer N,N′-dicyclohexyl-p-phenylenediamine is distinctly reduced.

The unit “phr” hereinafter stands for parts by weight based on 100 parts by weight of the total amount of rubber present in the rubber mixture, i.e. the total amount of natural rubber(s) and any synthetic rubbers present.

The present invention provides rubber mixtures comprising at least one natural rubber and N,N′-dicyclohexyl-p-phenylenediamine.

The rubber mixtures of the invention comprise N,N′-dicyclohexyl-p-phenylenediamine in an amount of 0.5 to 4.0, preferably of 0.5 to 3.0 and more preferably of 0.5 to 2.5 phr.

The rubber mixtures of the invention comprise at least one natural rubber.

Natural rubber is a rubberlike substance that occurs in the latex of many different rubber plants. This is a polymer of the monomer isoprene (2-methyl-1,3-butadiene) with an almost uniform cis-1,4 linkage. The average molar mass of natural rubber is about 500,000 to 2 million g·mol⁻¹.

Natural rubber composed of cis-1,4-polyisoprene is obtained mainly from the sap of the bark of Hevea basiliensis or of Landolphia owariensis, from the Mexican guayule shrub or the Russian dandelion plant (taraxacum koksaghyz). In addition, there is trans-1,4-polyisoprene. This is obtained from the palaquium tree (Palaquium gutta).

Likewise consisting of trans-1,4-polyisoprene is balata, which has a high resin content and is obtained from the balata tree (Manilkara bidentata).

According to the invention, it is possible to use all kinds of natural rubber and mixtures thereof. Preferred natural rubbers are, for example, technically specified natural rubber (TSR), for example Standard Malaysian rubber (SMR) and Ribbed Smoked Sheets (RSS).

In an alternative embodiment, the rubber mixtures of the invention, apart from the natural rubbers, also comprise one or more synthetic rubbers.

Preferred polar and nonpolar synthetic rubbers are, for example,
BR—polybutadiene
ABR—butadiene/C1-C4-alkyl acrylate copolymer
CR—polychloroprene
IR—polyisoprene
SBR—styrene/butadiene copolymers having styrene contents of 1-60%, preferably 20-50%, by weight
IIR—isobutylene/isoprene copolymers
NBR—butadiene/acrylonitrile copolymers having acrylonitrile contents of 5-60%, preferably 10-50%, by weight
HNBR—partially or fully hydrogenated NBR rubber
EPDM—ethylene/propylene/diene copolymers
SIBR—styrene-isoprene-butadiene rubber
ENR—epoxidized natural rubber
SNBR—acrylonitrile-styrene/butadiene rubber
HNBR—hydrogenated acrylonitrile/butadiene rubber
XNBR—carboxylated acrylonitrile/butadiene rubber
HXNBR—hydrogenated carboxylated acrylonitrile/butadiene rubber.

In an alternative embodiment, the rubber mixtures of the invention, apart from the natural rubbers, also comprise one or more nonpolar synthetic rubbers, preferably selected from the group consisting of SBR, BR, IR, SIBR, IIR, ENR and EPDM, more preferably from the group consisting of SBR, BR, IIR and EPDM, most preferably BR and/or SBR, where the total content of these nonpolar rubbers in the rubber mixture is generally 10 to 90 phr, preferably 20 to 80 phr and more preferably 30 to 60 phr.

The rubber mixtures of the invention may comprise one or more fillers. In principle, suitable fillers are all that are known from the prior art for this purpose, preference being given to the active or reinforcing fillers.

The rubber mixtures of the invention comprise generally 0.1 to 200 phr, preferably 20 to 160 phr and more preferably 25 to 140 phr of at least one filler.

The rubber mixtures of the invention preferably comprise at least one oxidic filler containing hydroxyl groups and/or at least one carbon black.

In general, the content of oxidic fillers containing hydroxyl groups in the rubber mixtures of the invention is 0.1 to 200 phr, preferably 20 to 160 phr, more preferably 25 to 140 phr and most preferably 30 to 120 phr.

Preferred oxidic fillers containing hydroxyl groups are preferably those from the group of
silicas having a specific surface area (BET) of 5 to 1000 and preferably 20 to 400 m²/g and having primary particle sizes of 100 to 400 nm, where the silicas are optionally also in the form of mixed oxides with other metal oxides, such as oxides of Al, Mg, Ca, Ba, Zn, Zr, Ti,
synthetic silicates, such as aluminum silicate, alkaline earth metal silicates such as magnesium silicate or calcium silicate, having specific surface areas (BET) of 20 to 400 m²/g and primary particle diameters of 10 to 400 nm,
   and
natural silicates, such as kaolin and other naturally occurring silicas, and mixtures thereof.

The oxidic fillers containing hydroxyl groups that are present in the rubber mixtures of the invention and are from the group of the silicas are preferably those that can be produced, for example, by precipitation of solutions of silicates or flame hydrolysis of silicon halides.

Preferably, the rubber mixtures of the invention comprise at least one oxidic filler containing hydroxyl groups from the group of the silicas having a specific surface area (BET) in the range from 20 to 400 ²/g in an amount of 0.1 to 200 phr, preferably 20 to 160 phr, more preferably of 25 to 140 phr, most preferably 30-120 phr.

All BET figures relate to the specific surface area measured to DIN 66131. The primary particle size figures relate to values ascertained by scanning electron microscope.

The rubber mixtures of the invention may further comprise at least one carbon black as filler.

The rubber mixtures of the invention preferably comprise at least one carbon black in an amount of 0.1 to 200 phr, preferably 20 to 160 phr, more preferably 25 to 140 phr, most preferably 30 to 120 phr.

Preference is given in accordance with the invention to carbon blacks that are obtainable by the lamp black, furnace black or gas black method and have a specific surface area (BET) in the range from 20 to 200 m²/g, for example SAF, ISAF, IISAF, HAF, FEF or GPF carbon blacks. The rubber mixtures of the invention preferably comprise at least one carbon black having a specific surface area (BET) in the range from 20 to 200 m²/g.

More preferably, fillers present in the rubber mixtures of the invention are at least one of the abovementioned carbon blacks and at least one of the abovementioned silicas.

The total amount of carbon black and silica-based fillers in the rubber mixture of the invention is preferably 40 to 320 phr, more preferably 50 to 280 phr and most preferably 60 to 240 phr.

The rubber mixtures of the invention may comprise one or more crosslinkers.

The rubber mixtures of the invention preferably comprise at least one crosslinker from the group of sulfur and sulfur donors and of the metal oxides, for example magnesium oxide and/or zinc oxide.

Sulfur may be used in elemental soluble or insoluble form.

More preferably, the rubber mixtures of the invention comprise at least one sulfur donor and/or sulfur, especially sulfur.

Examples of useful sulfur donors include dimorpholyl disulfide (DTDM), 2-morpholinodithiobenzothiazole (MBSS), caprolactam disulfide, dipentamethylenethiuram tetrasulfide (DPTT), tetramethylthiuram disulfide (TMTD) and tetrabenzylthiuram disulfide (TBzTB).

Most preferably, the rubber mixtures of the invention comprise tetrabenzylthiuram disulfide (TBzTB) as sulfur donor.

The rubber mixtures of the invention comprise generally 0.1 to 20 phr, preferably 0.5 to 10 phr and more preferably 1.0 to 8 phr of at least one of the crosslinkers mentioned.

The rubber mixtures of the invention may comprise one or more vulcanization accelerators.

The rubber mixtures of the invention preferably comprise at least one vulcanization accelerator, more preferably from the group of the mercaptobenzothiazoles, thiurams, thiocarbamates, dithiocarbamates, thiazoles, sulfenamides, thiazolesulfenamides, xanthogenates, bi- or polycyclic amines, thiophosphates, dithiophosphates, caprolactams, thiourea derivatives, guanidines, cyclic disulfanes and amines, especially zinc diaminediisocyanate, hexamethylenetetramine, 1,3-bis(citraconimidomethyl)benzene, and most preferably from the group of the sulfenamides, most preferably N-cyclohexylbenzothiazolesulfenamide (CAS No.: 95-33-0).

The rubber mixtures of the invention comprise generally 0.1 to 20 phr, preferably 0.5 to 10 phr and more preferably 1.0 to 5 phr of at least one of the vulcanization accelerators mentioned.

The rubber mixtures of the invention preferably comprise at least one crosslinker and at least one vulcanization accelerator.

More preferably, the rubber mixtures of the invention comprise at least one crosslinker from the group of sulfur, zinc oxide and magnesium oxide, and at least one vulcanization accelerator from the group of the mercaptobenzothiazoles, thiazolesulfenamides, thiurams, dithiocarbamates, xanthogenates and thiophosphates, more preferably from the sulfenamides, most preferably N-cyclohexylbenzothiazolesulfenamide (CAS No.: 95-33-0).

The crosslinkers and vulcanization accelerators are preferably used in the rubber mixtures of the invention in amounts of 0.1 to 15 phr, more preferably of 2.0 to 10 phr, based in each case on the sum total of these components.

The rubber mixtures of the invention may comprise one or more reinforcing additives.

The rubber mixtures of the invention preferably comprise at least one reinforcing additive from the group of the sulfur-containing organic silanes, especially the sulfur-containing silanes containing alkoxysilyl groups, and most preferably from the sulfur-containing organic silanes containing trialkoxysilyl groups.

More preferably, the rubber mixtures of the invention comprise one or more sulfur-containing silanes from the group of bis(triethoxysilylpropyl)tetrasulfane, bis(triethoxysilylpropyl)disulfane and 3-(triethoxysilyl)-1-propanethiol. The rubber mixtures of the invention comprise generally 0.1 to 20 phr, preferably 0.5 to 15 phr and more preferably 1.0 to 10 phr of at least one reinforcing additive.

Liquid sulfur-containing silanes may be absorbed on a carrier (dry liquid) for better meterability and/or dispersibility. The content of sulfur-containing silanes in these dry liquids is preferably between 30 and 70 parts by weight, preferably 40 and 60 parts by weight, per 100 parts by weight of dry liquid.

The rubber mixtures of the invention may further comprise one or more rubber auxiliaries. Examples of useful rubber auxiliaries include aging stabilizers, bonding agents, heat stabilizers, light stabilizers, flame retardants, processing aids, impact resistance improvers, plasticizers, tackifiers, blowing agents, dyes, pigments, waxes, extenders, organic acids, retardants, metal oxides and activators, especially triethanolamine, polyethylene glycol, hexanetriol and reversion stabilizers.

These rubber auxiliaries may be added to the rubber mixtures of the invention in the amounts that are customary for these auxiliaries, which are also guided by the end use of the vulcanizates produced therefrom. Customary amounts are, for example, 0.1 to 30 phr.

Apart from the N, N′-dicyclohexyl-p-phenylenediamine, the rubber mixtures of the invention may also comprise one or more further aging stabilizers. Suitable aging stabilizers are aminic aging stabilizers, for example mixtures of diaryl-p-phenylenediamines (DTPD), octylated diphenylamine (ODPA), phenyl-α-naphthylamine (PAN), phenyl-β-naphthylamine (PBN), preferably those based on phenylenediamine, e.g. N-isopropyl-N′-phenyl-p-phenylenediamine, N-1,3-dimethylbutyl-N′-phenyl-p-phenylenediamine (6PPD), N-1,4-dimethylpentyl-N′-phenyl-p-phenylenediamine (7PPD), N,N′-bis(1,4-dimethylpentyl)-p-phenylenediamine (77PD), and phosphites such as tris(nonylphenyl) phosphite, polymerized 2,2,4-trimethyl-1,2-dihydroquinoline (TMQ), methyl-2-mercaptobenzimidazole (MMBI) and zinc methylmercaptobenzimidazole (ZMMBI).

Processing aids should be active between the rubber particles and should counter frictional forces during mixing, plasticizing and forming. Processing aids which may be present in the rubber mixtures according to the invention include all lubricants customary for the processing of plastics, for example hydrocarbons, such as oils, paraffins and PE waxes, fatty alcohols having 6 to 20 carbon atoms, ketones, carboxylic acids, such as fatty acids and montanic acids, oxidized PE wax, metal salts of carboxylic acids, carboxamides and carboxylic esters, for example with the alcohols ethanol, fatty alcohols, glycerol, ethanediol, pentaerythritol and long-chain carboxylic acids as the acid component.

To reduce flammability and to reduce smoke evolution on combustion, the rubber mixtures of the invention may also comprise flame retardants. Examples of compounds used for this purpose include antimony trioxide, phosphoric esters, chloroparaffin, aluminum hydroxide, boron compounds, zinc compounds, molybdenum trioxide, ferrocene, calcium carbonate and magnesium carbonate.

Further plastics may also be added to the rubber mixtures of the invention prior to the crosslinking, these acting for example as polymeric processing aids or impact modifiers. These plastics are preferably selected from the group consisting of homo- and copolymers based on ethylene, propylene, butadiene, styrene, vinyl acetate, vinyl chloride, glycidyl acrylate, glycidyl methacrylate, acrylates and methacrylates having alcohol components of branched or unbranched C1 to C10 alcohols, particular preference being given to polyacrylates having identical or different alcohol radicals from the group of C4 to C8 alcohols, in particular of butanol, hexanol, octanol and 2-ethylhexanol, polymethylmethacrylate, methyl methacrylate-butyl acrylate copolymers, methyl methacrylate-butyl methacrylate copolymers, ethylene-vinyl acetate copolymers, chlorinated polyethylene, ethylene-propylene copolymers, ethylene-propylene-diene copolymers.

Known bonding agents are based on resorcinol, formaldehyde and silica, the so-called RFS direct adhesion systems. These direct bonding systems may be used in the rubber mixture of the invention in any desired amount at any time during incorporation into the rubber mixtures of the invention.

In silica-based rubber mixtures as used for tire production, diphenylguanidine (DPG) or structurally similar aromatic guanidines are typically used as secondary accelerators for controlled adjustment of the crosslinking rate and the mixture viscosity within the mixing process. However, a very important adverse feature associated with the use of DPG is that it releases aniline during vulcanization, which is suspected to be carcinogenic. It is now been found that, surprisingly, in the rubber mixtures of the invention, DPG can advantageously be replaced by 1,6-bis(N,N-dibenzylthiocarbamoyldithio)hexane (trade name: Vulcuren®). Replacement of DPG by secondary accelerators such as TBzTD (tetrabenzylthiuram disulfide) or dithiophosphates is also possible.

The present invention therefore also encompasses essentially DPG-free rubber mixtures.

The rubber mixtures of the invention preferably comprise at least one secondary accelerator from the group of 1,6-bis(N,N-dibenzylthiocarbamoyldithio)hexane (trade name: Vulcuren®), tetrabenzylthiuram disulfide (TBzTD) and dithiophosphates.

The rubber mixtures of the invention comprise generally 0.1 to 5.0 phr, preferably 0.2 to 2.5 phr, of at least one of the secondary accelerators mentioned.

The present invention therefore also provides rubber mixtures of the invention that are essentially free of diphenylguanidine and/or substituted diphenylguanidines, especially those having a content of diphenylguanidine and/or substituted diphenylguanidines of not more than 0.4 phr, preferably of 0.1 to 0.2 phr, more preferably of 0.05 to 0.1 phr and most preferably of 0.001 to 0.04 phr.

Preference is given to rubber mixtures of the invention comprising
at least one natural rubber,
20 to 160 phr of at least one silica, especially having a specific surface area (BET) of 5 to 1000 and preferably 20 to 400 m²/g and having primary particle sizes of 100 to 400 nm,
   and/or
20 to 160 phr of at least one carbon black, especially having a specific surface area (PET) in the range from 20 to 200 m²/g,
   and
0.5 to 4.0 phr of N,N′-dicyclohexyl-p-phenylenediamine.

Particular preference is given to rubber mixtures of the invention comprising
at least one natural rubber,
25 to 140 phr of at least one silica, especially having a specific surface area (BET) of 5 to 1000 and preferably 20 to 400 m²/g and having primary particle sizes of 100 to 400 nm,
25 to 140 phr of at least one carbon black, especially having a specific surface area (PET) in the range from 20 to 200 m²/g,
0.5 to 20 phr of at least one crosslinker, especially from the group of the sulfur donors and/or sulfur,
0.5 to 20 phr of at least one vulcanization accelerator, especially from the group of the sulfenamides,
1.0 to 20 phr of at least one reinforcing additive, especially from the group of the sulfur-containing silanes,
   and
0.5 to 4.0 phr of N,N′-dicyclohexyl-p-phenylenediamine.

In an alternative embodiment, preference is likewise given to rubber mixtures of the invention comprising at least one natural rubber,
5 to 100 phr of at least one synthetic rubber, especially BR rubber and/or SBR rubber,
20 to 160 phr of at least one silica, especially having a specific surface area (BET) of 5 to 1000 and preferably 20 to 400 m²/g and having primary particle sizes of 100 to 400 nm,
20 to 160 phr of at least one carbon black, especially having a specific surface area (PET) in the range from 20 to 200 m²/g, and
0.5 to 4.0 phr of N,N′-dicyclohexyl-p-phenylenediamine.

In an alternative embodiment, particular preference is likewise given to rubber mixtures of the invention comprising
at least one natural rubber,
5 to 100 phr of at least one synthetic rubber, especially BR rubber and/or SBR rubber,
20 to 160 phr of at least one silica, especially having a specific surface area (BET) of 5 to 1000 and preferably 20 to 400 m²/g and having primary particle sizes of 100 to 400 nm,
20 to 160 phr of at least one carbon black, especially having a specific surface area (PET) in the range from 20 to 200 m²/g,
0.5 to 20 phr of at least one crosslinker, especially from the group of the sulfur donors and/or sulfur,
0.5 to 20 phr of at least one vulcanization accelerator, especially from the group of the sulfenamides,
1.0 to 20 phr of at least one reinforcing additive, especially from the group of the sulfur-containing silanes, and
0.5 to 4.0 phr of N,N′-dicyclohexyl-p-phenylenediamine.

The present invention further provides a process for producing the rubber mixtures of the invention, characterized in that at least one natural rubber and N,N′-dicyclohexyl-p-phenylenediamine, optionally in the presence of at least one synthetic rubber, optionally at least one filler, optionally at least one crosslinker, optionally at least one vulcanization accelerator, optionally at least one reinforcing additive and optionally one or more of the abovementioned rubber auxiliaries, in the general and preferred amounts specified for these additives, are mixed with one another at a temperature in the range from 40 to 150° C., more preferably from 40 to 120° C.

The rubber mixtures of the invention are produced in a customary manner in known mixing apparatuses, such as rollers, internal mixers, downstream mixing roll systems and mixing extruders, at shear rates of 1 to 1000 sec⁻¹.

It is customary to employ a two-stage mixing process. This involves first incorporating the fillers and the N,N′-dicyclohexyl-p-phenylenediamine and any further abovementioned additives into the rubber in an internal mixer (kneader). Mixing temperatures in the internal mixer may reach values above 120° C. Therefore, the crosslinkers such as sulfur, zinc oxide and the vulcanization accelerators are mixed in at low temperatures, preferably at 30 to 100° C., in order to avoid incipient vulcanization.

The mixing-in can be effected in a roller system, wherein the large-area rolls, the temperature of which is controlled with water, enable a significantly lower mixing temperature.

The N,N′-dicyclohexyl-p-phenylenediamine can in principle be added at any time during the mixing, preferably in the first step of the mixing operation at a temperature in the range from 40° C. to 150° C., preferably at a temperature of <50° C.

The N,N′-dicyclohexyl-p-phenylenediamine is preferably added prior to addition of the crosslinker and the vulcanization accelerator, in the first mixing step. The N,N′-dicyclohexyl-p-phenylenediamine may be used either in pure form or else having been absorbed and/or adsorbed in the mixing process on an inert, organic or inorganic carrier, preferably a carrier selected from the group comprising natural and synthetic silicates, in particular neutral, acidic or basic silica, aluminum oxide, carbon black and zinc oxide.

The rubber mixtures of the invention and rubber vulcanizates produced therefrom may be used advantageously either in zinc-free or in zinc-containing rubber vulcanizates.

The present invention further relates to a process for producing rubber vulcanizates by heating a rubber mixture of the invention at melt temperatures of 150 to 200° C., preferably at 160 to 180° C. The process for producing the rubber vulcanizates of the invention can be conducted within a wide pressure range; it is preferably conducted at a pressure in the range from 10 to 200 bar.

The present invention further provides rubber vulcanizates obtainable by vulcanization of a rubber mixture of the invention.

The rubber vulcanizates of the invention, especially when used in tires, have the benefits of an excellent profile of properties and unexpectedly low rolling resistance.

The present invention further provides bonding mixtures comprising a rubber mixture of the invention and at least one bonding agent.

The bonding mixtures of the invention preferably comprise at least one bonding agent based on resorcinol, formaldehyde and silica.

Combinations of resorcinol, formaldehyde and silica are known from the prior art as RFS direct bonding systems. The bonding mixtures of the invention may comprise these direct bonding systems in any amount.

The bonding mixtures of the invention may be produced in a known manner by mixing a rubber mixture of the invention with at least one bonding agent based on resorcinol, formaldehyde and silica.

In the bonding agents, formaldehyde may be present in the form of formaldehyde donors. Suitable formaldehyde donors include not only hexamethylenetetramine but also methylolamine derivatives.

In order to improve bonding, one or more components capable of synthetic resin formation, such as phenol and/or amines and/or aldehydes and/or compounds that eliminate aldehydes, may be added to the bonding mixtures of the invention.

The rubber vulcanizates of the invention are suitable for production of all kinds of shaped bodies, for example tire components, industrial rubber articles such as damping elements, roll coverings, coverings of conveyor belts, drive belts, spinning cops, seals, golfball cores, footwear soles; they are especially suitable for production of tires and tire components, such as tire treads, subtreads, carcasses, sidewalls of tires, reinforced sidewalls for runflat tires, and apex mixtures. Tire treads here also include treads of summer, winter and all-season tires, and treads of car and truck tires.

The present invention provides moldings, especially tires and tire parts, comprising a rubber vulcanizate of the invention.

The present invention further provides for the use of N,N′-dicyclohexyl-p-phenylenediamine as aging stabilizer for vulcanizates based on natural rubber.

The invention is to be elucidated by the examples that follow, but without being limited thereto.

### WORKING EXAMPLES

### List of Feedstocks, Abbreviations and Manufacturers

| | | Manufacturer/ |
|---|---|---|
| Trade name | Description | distributor |
| TSR/RSS 3 | Natural rubber (NR) | Weber & |
| (premasticated to DEFO | | Schaer |
| hardness 1000) | | GmbH&Co |
| CORAX ® N 220 | Carbon black | Degussa- |
| | | Evonik |
| | | GmbH |
| VIVATEC 500 | Process oil | Hansen & |
| | | Rosenthal |
| EDENOR ® C 18 98 MY | Stearic acid | Cognis |
| | | Deutschland |
| | | GmbH |
| ZINKOXYD AKTIV ® | Zinc oxide | Lanxess |
| | | Deutschland |
| | | GmbH |
| VULKACIT ® CZ/C | N-Cyclohexyl-2-benzothiazole-sulfenamide | Lanxess |
| | | Deutschland |
| | | GmbH |
| MAHLSCHWEFEL | Sulfur | Solvay |
| 90/95 CHANCEL | | Deutschland |
| | | GmbH |
| VULKANOX ® | N-1,3-Dimethylbutyl-N′-phenyl-p- | Lanxess |
| 4020/LG | phenylenediamine | Deutschland |
| | | GmbH |
| VULKANOX ® 4060 | N,N′-Dicyclohexyl-p-phenylenediamine | Lanxess |
| | (CAS No. 4175-38-6) | Deutschland |
| | | GmbH |
| BUNA ® CB 24 | Polybutadiene rubber (BR) | Arlanxeo |
| BUNA ® VSL 4526-2 | Styrene-butadiene rubber (SBR) | Arlanxeo |
| HM | | |
| CORAX ® N 339 | Carbon black | Degussa- |
| | | Evonik |
| | | GmbH |
| VULKASIL ® S | Silica | Lanxess |
| | | Deutschland |
| | | GmbH |
| TUDALEN ® 1849-TE | Process oil | Hansen & |
| | | Rosenthal |
| PALMERA ® A9818 | Stearic acid | KLK Oleo |
| AVOZINC ® Rotsiegel/ | Zinc oxide | Avokal- |
| ZINKOXID | | Heller |
| ROTSIEGEL | | |
| ANTILUX ® 654 | Wax | Lanxess |
| | | Deutschland |
| | | GmbH |
| SI 69 ® | Silane (bis{3- | Evonik |
| | (triethoxysilyl)propyl}tetrasulfane (TESPT)) | Operations |
| | | GmbH |
| RHENOGRAN ® DPG- | 80% diphenylguanidine in masterbatch form | Lanxess |
| 80 | | Deutschland |
| | | GmbH |
| TSR/RSS 3 DEFO 700 | Natural rubber | Handelshaus |
| | | Weber |
| | | & Schaer |
| CORAX ® N 326 | Carbon black | Degussa- |
| | | Evonik |
| | | GmbH |
| VULKANOX ® HS | Polymerized 2,2,4-trimethyl-1,2- | Lanxess |
| | dihydroquinoline | Deutschland |
| | | GmbH |
| COHEDUR ® A 250 | Melamine formaldehyde ether as | Lanxess |
| | condensation product from melamine, | Deutschland |
| | formaldehyde, methanol on filler (50%) | GmbH |
| COHEDUR ® RS | Resorcinol and stearic acid (2:1) | Lanxess |
| | | Deutschland |
| | | GmbH |
| KORESIN ® PULVER | Tackifier resin | Schill & |
| | | Seilacher |
| VULKACIT ® DZ | Dicyclohexylbenzothiazyl-sulfenamide | Lanxess |
| | | Deutschland |
| | | GmbH |

### Production of the Rubber Vulcanizates

### Examples 1-3

The rubber mixtures of noninventive examples 1 and 2 and of inventive example 3 were produced according to the formulations specified in table 1a.

Examples 1 to 3 differ merely in that no aging stabilizer was used in example 1, while the known VULKANOX® 4020/LG was used in example 2, and N,N′-dicyclohexyl-p-phenylenediamine in inventive example 3.

For this purpose, in each case, in a first mixing step, a kneader (GK 1.5) was additionally charged with the natural rubber, and the additives CORAX® N 220, VIVATEC 500, EDENOR® C 18 98 MY and, in example 2, the aging stabilizer VULKANOX® 4020/LG, and, in example 3, N,N′-dicyclohexyl-p-phenylenediamine were mixed at a temperature of 110° C. and about 40 revolutions per second, and then, in a second mixing step, the rubber mixtures thus produced were each applied to a temperature-controlled roller and the further additives ZINKOXYD AKTIV®, MAHLSCHWEFEL 90/95 CHANCEL and VULKACIT® CZ/C were added and incorporated into the rubber mixtures. The roller temperature was 40° ° C.

The rubber mixtures thus produced were then fully vulcanized at 150° ° C. and rolled to test plaques of thickness about one centimeter.

These test plaques were used for the subsequent performance tests as specified below.

TABLE 1a
Rubber formulations

| Example | 1 | 2 | 3 |
|---|---|---|---|
| Inventive yes/no | no | no | yes |
| Natural rubber TSR/RSS 3 | 100 | 100 | 100 |
| DEFO 1000 | | | |
| CORAX ® N 220 | 48 | 48 | 48 |
| VIVATEC 500 | 2.5 | 2.5 | 2.5 |
| EDENOR ® C 18 98 MY | 3 | 3 | 3 |
| ZINKOXYD AKTIV ® | 5 | 5 | 5 |
| VULKACIT ® CZ/C | 1.5 | 1.5 | 1.5 |
| MAHLSCHWEFEL 90/95 | 1.5 | 1.5 | 1.5 |
| CHANCEL | | | |
| VULKANOX ® 4020/LG | 0 | 1 | 0 |
| VULKANOX ® 4060 | 0 | 0 | 1 |

### Examples 4 and 5

The rubber mixtures of noninventive example 4 and of inventive example 5 were produced according to the formulations specified in table 1b.

Examples 4 and 5 differ merely in that, in example 4, VULKANOX®4020/LG (N-1,3-dimethylbutyl-N′-phenyl-p-phenylenediamine) was used and, in example 5, VULKANOX® 4060 (N,N′-dicyclohexyl-p-phenylenediamine) was used instead.

The rubber mixtures of examples 4 and 5 were produced in the following steps:

### 1st Mixing Stage:

BUNA® CB 24 and BUNA® VSL 4526-2 HM are initially charged in an internal mixer and mixed for about 30 seconds.
Add two thirds of VULKASIL® S, two thirds of SI® 69, mix for about 60 seconds
Add one third of VULKASIL® S, one third of SI® 69, and TUDALEN® 1849-TE, mix for about 60 seconds

Add CORAX® N 339, PALMERA® A9818, VULKANOX® 4020/LG or Vulkanox® 4060, VULKANOX® HS, ZINKOXID (ROTSIEGEL) and ANTILUX® 654, mix for about 60 seconds. This mixing operation was effected at 110° C.

On conclusion of the first mixing stage, the respective mixing batch was accepted by a downstream roll mill and shaped to a plaque and stored at room temperature for 24 hours.

Processing temperatures here are 55° C.

Further mastication is effected at 150° C. in a kneader/internal mixer.

### 2nd Mixing Stage:

Addition of additives, for example MAHLSCHWEFEL 90/95 CHANCEL, VULKACIT® CZ/C, RHENOGRAN® DPG-80, on a roll at a temperature of 75° C.

These test plaques were used for the subsequent performance tests as specified below.

TABLE 1b
Rubber formulations comprising sulfur-containing silanes

| Example | 4 | 5 |
|---|---|---|
| Inventive yes/no | no | yes |
| BUNA ® CB 24 | 30 | 30 |
| BUNA ® VSL 4526-2 HM | 96 | 96 |
| CORAX ® N 339 | 6.4 | 6.4 |
| VULKASIL ® S | 80 | 80 |
| TUDALEN ® 1849-TE | 8 | 8 |
| PALMERA ® A9818 | 1 | 1 |
| VULKANOX ® HS | 1 | 1 |
| ZINKOXID ROTSIEGEL | 2.5 | 2.5 |
| ANTILUX ® 654 | 1.5 | 1.5 |
| SI 69 ® | 6.4 | 6.4 |
| VULKACIT ® CZ/C | 1.5 | 1.5 |
| RHENOGRAN ® DPG-80 | 2.5 | 2.5 |
| MAHLSCHWEFEL 90/95 | 1.5 | 1.5 |
| CHANCEL | | |
| VULKANOX ® 4020/LG | 1 | 0 |
| VULKANOX ® 4060 | 0 | 1 |

### Examples 6 and 7

The bonding mixtures of noninventive example 6 and of inventive example 7 were produced according to the formulations specified in table 1c.

TABLE 1c
Formulations of bonding mixtures 1 and 2

| | Example 6 | Example 7 |
|---|---|---|
| | Bonding mixture 1 | Bonding mixture 2 |
| Inventive yes/no | no | yes |
| VULKASIL ® S | 12.7 | 12.7 |
| COHEDUR ® A 250 | 4.6 | 4.6 |
| COHEDUR ®RS | 3.4 | 3.4 |
| VULKANOX ® HS | 2.0 | 0 |
| VULKANOX ® 4060 | 0 | 2.0 |
| TSR/RSS 3 DEFO 700 | 100 | 100 |
| CORAX ® N 326 | 50 | 50 |
| TUDALEN ® 1849-TE | 4 | 4 |
| ZINKOXYD AKTIV ® | 8 | 8 |
| KORESIN ®-PULVER | 4 | 4 |
| MAHLSCHWEFEL 90/95 | 4.5 | 4.5 |
| CHANCEL | | |
| VULKACIT ® DZ | 0.7 | 0.7 |

For the production of bonding mixtures 1 and 2, in a first step, VULKASIL® S was first admixed with COHEDUR® A 250, and then contacted with COHEDUR® RS, with the use amounts as in table 1c. Thereafter, bonding mixture 1 was admixed with VULKANOX® HS, and bonding mixture 2 with VULKANOX® 4060. The mixtures thus obtained are referred to hereinafter as base bonding mixtures 1 and 2.

In a second step, in an internal mixer, the following are added successively to the natural rubber: base bonding mixture 1 in example 6, or base bonding mixture 2 in example 7, followed by carbon black and mineral oil. The internal mixer had a temperature of 90° C. and the residence time of the bonding components was 4 minutes and 40 seconds.

In the roll mill, the following were subsequently added to each of the two rubber mixtures: VULKACIT® DZ and MAHLSCHWEFEL 90/95 CHANCEL, and the other constituents for bonding mixtures 1 and 2 in the amounts specified in table 1c.

The mixing roll mill was at a temperature of 40° C. The milled sheets thus obtained were used for the measurement of full vulcanization time (t95).

A further portion of the mixtures was vulcanized in an electric heating press. The crosslinking temperature was T=150° C. The rubber vulcanizates thus obtained were used for the measurements of elongation at break and tensile strength.

### Technical Testing

The vulcanizates produced from the rubber mixtures of examples 1 to 3 and 4 to 7 were subjected to the technical tests specified below. The values ascertained can be found in tables 2 and 3.

### Rheometer (Vulcameter) Used and Full Vulcanization Time

The MDR (moving die rheometer) vulcanization profile and analytical data associated therewith were measured in an MDR 2000 Monsanto rheometer in accordance with ASTM D5289-95.

The full vulcanization time (t95) determined is the time at which 95% of the rubber has been crosslinked. The temperature chosen was 150° C.

### Determination of Elongation at Break and Tensile Strength

These measurements were effected in accordance with DIN 53504 (tensile test, S 2 specimen) after 0, 7, 14 and 28 days, in relation to examples 1-3. The test specimens were stored at a temperature of 60° C.

In examples 4-7, the measurements were in accordance with DIN 53504 (tensile test, S 2 specimen) after day 0.

TABLE 2
Elongation at break and tensile strength

| Example | 1 | 2 | 3 | 4 | 5 | 6 | 7 |
|---|---|---|---|---|---|---|---|
| Inventive | no | no | yes | no | yes | no | yes |
| yes/no | | | | | | | |
| Full | 691 | 663 | 467 | 1157 | 995 | 2475 | 2325 |
| vulcanization | | | | | | | |
| time (t95) [s] | | | | | | | |
| Elongation at | 401 | 427 | 477 | 356 | 380 | 462 | 512 |
| break [%] | | | | | | | |
| Tensile | 27.5 | 28.8 | 31.5 | 20.1 | 20.1 | 23.4 | 24.9 |
| strength [MPa] | | | | | | | |

TABLE 2.1
Elongation at break values as a function of time

| | Example | | |
|---|---|---|---|
| | 1 | 2 | 3 |
| | Elongation at | Elongation at | Elongation at |
| Days | break [%] | break [%] | break [%] |
| 0 d | 401 | 427 | 477 |
| 7 d | 379 | 406 | 426 |
| 14 d | 359 | 399 | 410 |
| 28 d | 317 | 361 | 379 |

TABLE 2.2
Tensile strength values as a function of time

| | Example | | |
|---|---|---|---|
| | 1 | 2 | 3 |
| Days | Tensile strength [%] | Tensile strength [%] | Tensile strength [%] |
| 0 d | 27.5 | 28.8 | 31.5 |
| 7 d | 25.9 | 28.2 | 29.5 |
| 14 d | 24.7 | 28.8 | 29.1 |
| 28 d | 21.3 | 26.7 | 27.5 |

### Determination of Volatility

The loss of mass over time was determined at 130° C. and converted to percent.

TABLE 3
Volatility in percent at 130° C. as a function of time

Example

| | 2 | 3 |
|---|---|---|
| Time (min) | Dry mass (% R) | Dry mass (% R) |
| 0.0 | 100.00 | 100.00 |
| 10.0 | 99.66 | 99.22 |
| 20.0 | 99.80 | 99.73 |
| 30.0 | 99.80 | 99.93 |
| 40.0 | 99.85 | 99.91 |
| 50.0 | 99.80 | 99.91 |
| 60.0 | 99.80 | 99.91 |
| 70.0 | 99.75 | 99.91 |
| 80.0 | 99.71 | 99.84 |
| 90.0 | 99.71 | 99.84 |
| 100.0 | 99.61 | 99.84 |
| 110.0 | 99.61 | 99.84 |
| 120.0 | 99.61 | 99.78 |
| 130.0 | 99.56 | 99.78 |
| 140.0 | 99.56 | 99.71 |
| 150.0 | 99.56 | 99.71 |
| 160.0 | 99.46 | 99.64 |
| 170.0 | 99.46 | 99.64 |
| 180.0 | 99.46 | 99.64 |
| 190.0 | 99.46 | 99.64 |
| 200.0 | 99.46 | 99.64 |
| 210.0 | 99.46 | 99.64 |
| 220.0 | 99.36 | 99.64 |
| 230.0 | 99.31 | 99.64 |
| 240.0 | 99.31 | 99.57 |

### CONCLUSION

It has been found that, surprisingly, with the inventive rubber mixture according to example 3, low full vulcanization times (t95) and distinctly higher values for elongation at break and tensile strength on storage at 60° C. over time are achievable by comparison with the noninventive rubber mixtures from example 1 (without aging stabilizer) and from example 2 (with N-1,3-dimethylbutyl-N′-phenyl-p-phenylenediamine (6PPD)). The rubber mixture of the invention does not show any specks on the surface, and so good mixing of the additives used can be assumed. In the processing of the inventive rubber mixture from example 3, less emission was observed than in the processing of the noninventive rubber mixture of example 2, as apparent from the values in table 3.

For the inventive rubber mixture from example 5 and the inventive bonding mixture from example 7, surprisingly, lower full vulcanization times (t95) and distinctly higher values for elongation at break and equal or higher values for tensile strength were achieved by comparison with the noninventive rubber mixture 4 or the noninventive bonding mixture from example 6.

1. A rubber mixture, characterized in that it comprises at least one natural rubber and N,N′-dicyclohexyl-p-phenylenediamine.

2. The rubber mixture as claimed in claim 1, characterized in that it comprises N,N′-dicyclohexyl-p-phenylenediamine in an amount of 0.5 to 4.0 phr, preferably of 0.5 to 3.0 phr and more preferably of 0.5 to 2.5 phr.

3. The rubber mixture as claimed in claim 1 or 2, characterized in that it comprises at least one oxidic filler containing hydroxyl groups in an amount of 0.1 to 200 phr, preferably 20 to 160 phr, more preferably 25 to 140 phr and most preferably 30 to 120 phr.

4. The rubber mixture as claimed in any of claims 1 to 3, characterized in that it comprises at least one carbon black in an amount of 0.1 to 200 phr, preferably of 20 to 160 phr, more preferably of 25 to 140 phr and most preferably of 30 to 120 phr.

5. The rubber mixture as claimed in any of claims 1 to 4, characterized in that it comprises at least one crosslinker, preferably from the group of sulfur, sulfur donors and metal oxides.

6. The rubber mixture as claimed in any of claims 1 to 5, characterized in that it comprises at least one vulcanization accelerator, preferably from the group of the mercaptobenzthiazoles, thiocarbamates, dithiocarbamates, thiurams, thiazoles, sulfenamides, thiazolesulfenamides, xanthogenates, bi- or polycyclic amines, thiophosphates, dithiophosphates, caprolactams, thiourea derivatives, guanidines, cyclic disulfanes and amines, especially zinc diaminediisocyanate, hexamethylenetetramine, 1,3-bis(citraconimidomethyl)benzene.

7. The rubber mixture as claimed in any of claims 1 to 6, characterized in that it comprises at least one reinforcing additive from the group of the sulfur-containing organic silanes, especially the sulfur-containing silanes containing alkoxysilane groups.

8. The rubber mixture as claimed in any of claims 1 to 7, characterized in that it comprises at least one secondary accelerator from the group of 1,6-bis(N,N-dibenzylthiocarbamoyldithio)hexane, tetrabenzylthiuram disulfide (TBzTD) and dithiophosphates.

9. The rubber mixture as claimed in any of claims 1 to 8, characterized in that it has a content of diphenylguanidine and/or substituted diphenylguanidines of not more than 0.4 phr, preferably of 0.1 to 0.2 phr, more preferably of 0.05 to 0.1 phr and most preferably of 0.001 to 0.04 phr.

10. The rubber mixture as claimed in any of claims 1 to 9, characterized in that it comprises one or more synthetic rubbers.

11. The use of N,N′-dicyclohexyl-p-phenylenediamine as aging stabilizer for rubber mixtures and rubber vulcanizates based on natural rubber.

12. A process for producing a rubber mixture as claimed in any of claims 1 to 10, characterized in that at least one natural rubber and N,N′-dicyclohexyl-p-phenylenediamine, optionally in the presence of at least one synthetic rubber, optionally at least one filler, optionally at least one crosslinker, optionally at least one vulcanization accelerator, optionally at least one reinforcing additive and optionally one or more rubber auxiliaries, are mixed with one another at a temperature in the range from 40 to 150° C.

13. A rubber vulcanizate obtainable by vulcanization of a rubber mixture as claimed in any of claims 1 to 10.

14. A process for producing a rubber vulcanizate, characterized in that at least one rubber mixture as claimed in any of claims 1 to 10 is heated to a temperature in the range from 150 to 200° C., preferably of 160 to 180° C.

15. A shaped body, especially tire, tire part or industrial rubber article, characterized in that it comprises at least one rubber vulcanizate as claimed in claim 13.

16. A bonding mixture comprising at least one rubber mixture as claimed in any of claims 1 to 10 and at least one bonding agent based on resorcinol, formaldehyde and silica.
